(19) United States
(12) Patent Application Publication
(10) Pub. No.: US 2026/0345591 A1
(43) Pub. Date: Nov. 5, 2026

(54) COMBINATIONS OF ESTROGEN RECEPTOR DEGRADERS AND CDK4 INHIBITORS

(71) Applicant: PFIZER INC., NEW YORK, NY (US)

(72) Inventors: Sudhakar Reddy CHINTHARLAPALLI, San Diego, CA (US); Julia Perkins SMITH, Darien, CT (US); Olga VALOTA, Legnano (MI) (IT)

(73) Assignee: PFIZER INC., NEW YORK, NY (US)

(21) Appl. No.: 19/487,941

(22) PCT Filed: May 30, 2024
(86) PCT No.: PCT/IB2024/055297

§ 371 Date: Nov. 25, 2025

Related U.S. Application Data
(60) Provisional application No. 63/470,764, filed on Jun. 2, 2023.
(60) Provisional application No. 63/609,846, filed on Dec. 13, 2023.
(60) Provisional application No. 63/617,661, filed on Jan. 4, 2024.
(60) Provisional application No. 63/568,410, filed on Mar. 21, 2024.

(51) Int. Cl. A61K 31/496; A61K 31/506; A61P 35/00; A61P 35/04

(52) U.S. Cl. CPC A61K 31/496; A61K 31/506; A61P 35/00; A61P 35/04

(57) ABSTRACT

Disclosed herein are methods for treating cancer comprising administering to a subject Compound A, or a pharmaceutically acceptable salt thereof, in combination with a selective CDK4 inhibitor.

### BACKGROUND OF THE INVENTION

Certain bifunctional compounds can target specific cellular proteins for degradation via the ubiquitin-proteasome system. Examples of such proteolysis targeting chimeric compounds (i.e., “PROTAC® protein degraders”) that target the Estrogen Receptor (ER) for ubiquitination and subsequent degradation are disclosed in International Publication No. WO 2018/102725, which is incorporated herein by reference in its entirety. Such bifunctional molecules exhibit a range of pharmacological activities consistent with the degradation of the ER including, but not limited to, treatment or amelioration of a disease condition such as cancer (e.g., breast cancer, uterine cancer, ovarian cancer, prostate cancer, endometrial cancer), or endometriosis.

A bifunctional molecule of particular interest is (S)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione or (3S)-3-[1,3-dihydro-1-oxo-5-[4-[[1-[4-[(1R,2S)-1,2,3,4-tetrahydro-6-hydroxy-2-phenyl-1-naphthalenylphenyl]-4-piperidinyl]methyl]-1-piperazinyl]-2H-isoindol-2-yl]-2,6-piperidinedione (referred to herein as “Compound A” or “Compd A”), which has the molecular formula of C₄₅H₄₉N₅O₄ and the following structure:

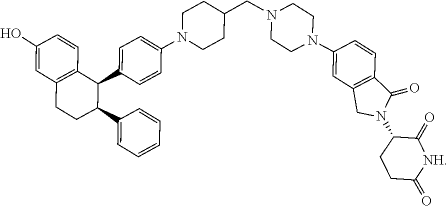 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Compound A is under development as a PROTAC® protein degrader that targets estrogen receptor (ER) for the potential treatment of breast cancer and has been shown to be a useful modulator of targeted protein ubiquitination and degradation via the ubiquitin-proteasome pathway.

There is a need for improved therapies for the treatment of cancer, such as, for example, an oral therapy for treating cancers (e.g., breast cancer).

### SUMMARY OF THE INVENTION

The present disclosure provides, in part, combinations and dosage regimens for administering Compound A, or a pharmaceutically acceptable salt thereof, to a subject, for treating cancer. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Provided herein are methods for treating cancer comprising administering to a subject Compound A having a structure of:

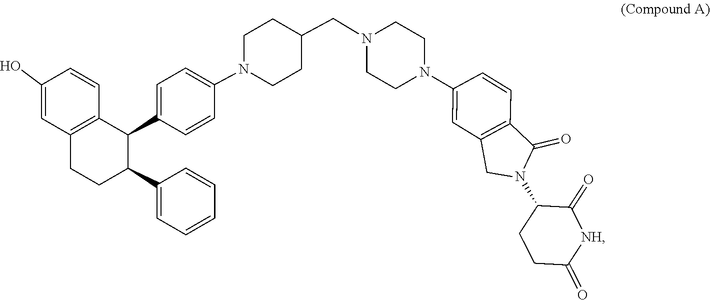 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, in combination with a selective CDK4 inhibitor.

In embodiments, a daily dose of Compound A, or a pharmaceutically acceptable salt thereof, is administered to the subject.

In embodiments, the daily dose of Compound A, or a pharmaceutically acceptable salt thereof, is about 200 mg.

In embodiments, the daily dose of Compound A, or a pharmaceutically acceptable salt thereof, is 200 mg.

In embodiments, the daily dose of Compound A, or a pharmaceutically acceptable salt thereof, is about 100 mg.

In embodiments, the daily dose of Compound A, or a pharmaceutically acceptable salt thereof, is 100 mg.

In embodiments, the daily dose of Compound A, or a pharmaceutically acceptable salt thereof, is administered once a day (QD).

In embodiments, the selective CDK4 inhibitor is administered concurrently or sequentially

In embodiments, Compound A, or a pharmaceutically acceptable salt thereof, is administered daily in 28-day cycles and the selective CDK4 inhibitor, is administered once a day or twice a day for each 28-day cycle. In embodiments, Compound B may be administered orally twice a day for each 28-day cycle.

In embodiments, the selective CDK4 inhibitor is administered intermittently

In embodiments, the daily dose of Compound A, or a pharmaceutically acceptable salt thereof, is administered orally to the subject.

In embodiments, the subject is in a fed state.

In embodiments, the selective CDK4 inhibitor is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof (referred to herein as “Compound B” or “Compd B”).

In embodiments, a daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is administered to the subject.

In embodiments, the daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is about 100 mg.

In embodiments, the daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is 100 mg.

In embodiments, the daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is about 300 mg.

In embodiments, the daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is 300 mg.

In embodiments, the daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is administered twice daily (BID).

In embodiments, a daily dose of Compound A, or a pharmaceutically acceptable salt thereof, and a daily dose of a selective CDK4 inhibitor is administered to the subject.

In embodiments, the daily dose of Compound A, or a pharmaceutically acceptable salt thereof, is about 200 mg QD; and the daily dose of a selective CDK4 inhibitor, which is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is about 100 mg BID.

In embodiments, the daily dose of Compound A, or a pharmaceutically acceptable salt thereof, is 200 mg QD; and the daily dose of a selective CDK4 inhibitor, which is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is 100 mg BID.

In embodiments, the daily dose of Compound A, or a pharmaceutically acceptable salt thereof, is about 200 mg QD; and the daily dose of a selective CDK4 inhibitor, which is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is about 300 mg BID.

In embodiments, the daily dose of Compound A, or a pharmaceutically acceptable salt thereof, is 200 mg QD; and the daily dose of a selective CDK4 inhibitor, which is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is 300 mg BID.

In embodiments, the daily dose of Compound A, or a pharmaceutically acceptable salt thereof, is about 100 mg QD; and the daily dose of a selective CDK4 inhibitor, which is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is about 100 mg BID.

In embodiments, the daily dose of Compound A, or a pharmaceutically acceptable salt thereof, is 100 mg QD; and the daily dose of a selective CDK4 inhibitor, which is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is 100 mg BID.

In embodiments, the daily dose of Compound A, or a pharmaceutically acceptable salt thereof, is about 100 mg QD; and the daily dose of a selective CDK4 inhibitor, which is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is about 300 mg BID.

In embodiments, the daily dose of Compound A, or a pharmaceutically acceptable salt thereof, is 100 mg QD; and the daily dose of a selective CDK4 inhibitor, which is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is 300 mg BID.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer. In embodiments, the breast cancer may be metastatic or locally advanced. In embodiments, the breast cancer may be estrogen receptor positive (ER+) breast cancer. Preferably, the breast cancer may be ER+ human epidermal growth factor receptor 2 negative (HER2−).

In embodiments, the subject is human.

It is to be understood that both the foregoing general description and the following detailed description are exemplary and explanatory only and are not restrictive of the invention, as claimed.

### BRIEF DESCRIPTION OF THE DRAWINGS

FIG. 1A shows a BLISS combination analysis of Compound B (30 mg/kg) alone and in combination with fulvestrant (10 mg/kg) on Day 41. Abbreviations: #: indicates synergistic effect of Compound B (30 mg/kg) alone versus Compound B (30 mg/kg) in combination with fulvestrant (10 mg/kg), p<0.05 versus BLISS; 10, 10 mg/kg; 30, 30 mg/kg; BID, twice a day; Fulv, fulvestrant; PF0060, Compound B; and SEM, standard error of the mean.

FIG. 1B shows a BLISS combination analysis of Compound B (60 mg/kg) alone and in combination with fulvestrant (10 mg/kg) on Day 41. Abbreviations: $ indicates synergistic effect of Compound B (60 mg/kg) alone versus Compound B (60 mg/kg) in combination with fulvestrant (10 mg/kg), p<0.05 vs. BLISS; 10, 10 mg/kg; 60, 60 mg/kg; BID, twice a day; Fulv, fulvestrant; PF0060, Compound B; and SEM, standard error of the mean.

FIG. 2A shows tumor growth inhibition by Compound B (30 mg/kg), Compound B (60 mg/kg) and fulvestrant (10 mg/kg) as single agents and in combinations. Abbreviations: 10, 10 mg/kg; 30, 30 mg/kg; 60, 60 mg/kg; BID, twice a day; Fulv, fulvestrant; PF0060, Compound B; and SEM, standard error of the mean.

FIG. 2B shows tumor growth inhibition by Compound A (30 mg/kg), Compound B (30 mg/kg), and Compound B (60 mg/kg) as single agents; and Compound A (30 mg/kg) in combination with Compound B (30 mg/kg), Compound B (60 mg/kg) and palbociclib (10 mg/kg). Abbreviations: 10, 10 mg/kg; 30, 30 mg/kg; 60, 60 mg/kg; BID, twice a day; Palbo, palbociclib; PF0060, Compound B; QD, once a day; and SEM, standard error of the mean.

FIG. 2C shows the tumor volume change on Day 41. Abbreviations: ΔTV, tumor volume change; % TGI, percent tumor growth inhibition; 10, 10 mg/kg; 30, 30 mg/kg; 60, 60 mg/kg; 4i, Compound B; BID, twice a day; CDK4i, Compound B; Fulv, fulvestrant; mono, monotherapy; Palbo, palbociclib; PF0060, Compound B; QD, once a day; SEM, standard error of the mean; veh, vehicle; and vs, versus.

FIG. 2D shows body weight change from Day 1 to Day 41. Abbreviations: 10, 10 mg/kg; 30, 30 mg/kg; 60, 60 mg/kg; BID, twice a day; Fulv, fulvestrant; Palbo, palbociclib; PF0060, Compound B; QD, once a day; SEM, standard error of the mean; and Wt, weight.

FIG. 3A shows tumor regrowth by Compound B (30 mg/kg), Compound B (60 mg/kg) and fulvestrant (10 mg/kg) as single agents and in combinations. Abbreviations: 10, 10 mg/kg; 30, 30 mg/kg; 60, 60 mg/kg; BID, twice a day; Fulv, fulvestrant; PF0060, Compound B; and SEM, standard error of the mean.

FIG. 3B shows tumor regrowth by Compound A (30 mg/kg), Compound B (30 mg/kg), and Compound B (60 mg/kg) as single agents; and Compound A (30 mg/kg) in combination with Compound B (30 mg/kg), Compound B (60 mg/kg) and palbociclib (10 mg/kg). Abbreviations: 10, 10 mg/kg; 30, 30 mg/kg; 60, 60 mg/kg; BID, twice a day; Palbo, palbociclib; PF0060, Compound B; QD, once a day; and SEM, standard error of the mean.

FIG. 3C shows body weight change from Day 1 to Day 41. Abbreviations: 10, 10 mg/kg; 30, 30 mg/kg; 60, 60 mg/kg; BID, twice a day; Fulv, fulvestrant; Palbo, palbociclib; PF0060, Compound B; QD, once a day; SEM, standard error of the mean; and Wt, weight.

### DETAILED DESCRIPTION OF THE INVENTION

The present invention may be understood more readily by reference to the following detailed description of the embodiments of the invention and the Examples included herein. It is to be also understood that the terminology used herein is for the purpose of describing specific embodiments only and is not intended to be limiting.

### Compound A:

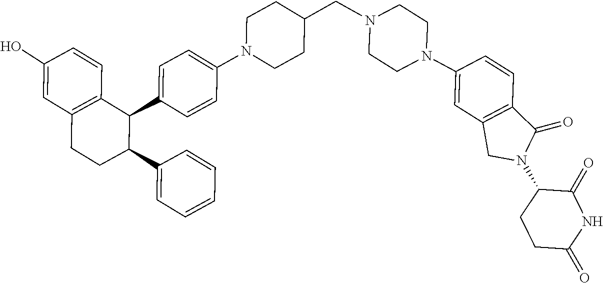 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is under development as a PROTAC® protein degrader that targets estrogen receptor (ER) for the potential treatment of breast cancer and has been shown to be a useful modulator of targeted protein ubiquitination and degradation via the ubiquitin-proteasome pathway. Compound A and pharmaceutically acceptable salts thereof are disclosed in International Publication No. WO 2018/102725 and U.S. Pat. Nos. 10,647,698, 10,899,742 and 11,104,666; International Publication No. WO 2021/041348; U.S. Ser. No. 17/472,847; U.S. Ser. No. 17/548,842; and U.S. Ser. No. 17/873,748. The contents of each of the foregoing references are incorporated herein by reference in their entirety.

Cyclin-dependent kinases (CDKs) and related serine/threonine protein kinases are important cellular enzymes that perform essential functions in regulating eukaryotic cell division and proliferation. The CDK catalytic units are activated by regulatory subunits known as cyclins. At least sixteen mammalian cyclins have been identified (Johnson D G, Walker C L. Cyclins and Cell Cycle Checkpoints. Annu. Rev. Pharmacol. Toxicol. (1999) 39:295-312). Cyclin B/CDK1, cyclin A/CDK2, cyclin E/CDK2, cyclin D/CDK4, cyclin D/CDK6, and likely other heterodynes are important regulators of cell cycle progression. Additional functions of cyclin/CDK heterodynes include regulation of transcription, DNA repair, differentiation, and apoptosis (Morgan D O, Cyclin-dependent kinases: engines, clocks, and microprocessors. Annu. Rev. Cell. Dev. Biol. (1997) 13:261-291).

CDK inhibitors have been demonstrated to be useful in treating cancer. Increased activity or temporally abnormal activation of cyclin-dependent kinases has been shown to result in the development of human tumors, and human tumor development is commonly associated with alterations in either the CDK proteins themselves or their regulators (Cordon-Cardo C. Mutations of cell cycle regulators: biological and clinical implications for human neoplasia. Am. J. Pathol. (1995) 147:545-560; Karp J E, Broder S. Molecular foundations of cancer: new targets for intervention. Nat. Med. (1995) 1:309-320; Hall M, Peters G. Genetic alterations of cyclins, cyclin-dependent kinases, and CDK inhibitors in human cancer. Adv. Cancer Res. (1996) 68:67-108).

CDK4 and CDK6 are important regulators of cell cycle progression at the G1-S checkpoint, which are controlled by D-type cyclins and INK4 endogenous CDK inhibitors, such as p16^{INK4a} (CDKN2A). Dysregulation of the cyclin D-CDK4/6-INK4-retinoblastoma (Rb) pathway has been reported to be associated with development of endocrine therapy resistance.

The use of CDK4/6 inhibitors in combination with endocrine therapy has demonstrated significant efficacy in the treatment of hormone receptor (HR)-positive, human epidermal growth factor 2 (HER2)-negative advanced or metastatic breast cancers, and CDK4/6 inhibitors, including palbociclib, ribociclib, and abemaciclib, have been approved in combination with endocrine therapy in a first- or second-line setting.

However, treatment with CDK4/6 inhibitors may result in adverse effects, such as gastrointestinal and/or hematologic toxicities, and acquired resistance may develop over time. Emerging data suggest that cyclin D3-CDK6 may be linked to the observed hematologic toxicity. (Malumbres et al., Mammalian Cells Cycle without the D-type Cyclin-Dependent Kinases Cdk4 and Cdk6, (2004) Cell 118(4):493-504; Sicinska et al. Essential Role for Cyclin D3 in Granulocyte Colony-Stimulating Factor-Driven Expansion of Neutrophil Granulocytes (2006), Mol. Cell Biol 26(21): 8052-60; Cooper et al. A unique function for cyclin D3 in early B cell development, (2006), Nat. Immunol. 5(7):489-97). CDK4 has been identified as the singular oncogenic driver in many breast cancers and emerging data suggest that cyclin D3-CDK6 inhibition may be linked to hematologic toxicity, suggesting a role for CDK4 selective inhibitors. Selective CDK4 inhibitors are selective for CDK4 over CDK6 and display greater CDK4-over-CDK6 selectivity as compared to dual CDK4/6 inhibitors. Accordingly, a CDK4 selective inhibitor may provide an improved safety profile or enhanced overall efficacy due to the potential of higher and/or continuous dosing compared to dual CDK4/6 inhibitors.

The compound 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (also referred to herein as “Compound B”) is a potent and selective inhibitor of CDK4, having the structure:

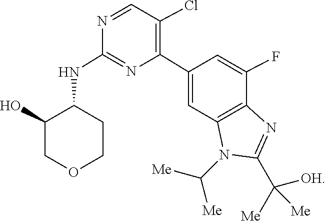 [SMILES: CC(C)n1c(C(C)(C)O)nc2c(F)cc(-c3nc(N[C@@H]4CCOC[C@H]4O)ncc3Cl)cc21]

Compound B and pharmaceutically acceptable salts thereof, are disclosed in International Publication No. WO 2019/207463 published Oct. 31, 2019, U.S. Pat. Nos. 10,766,884 and 11,220,494, and US Patent Publication US 2022/0089580; and International Publication No. WO 2022/058871 published Mar. 24, 2022, the contents of which are incorporated herein by reference in their entirety. Unless indicated otherwise, all references herein to Compound B include references to salts, solvates, hydrates, and complexes thereof, and to solvates, hydrates and complexes of salts thereof, including polymorphs, stereoisomers, and isotopically labelled versions thereof.

The methods of the present invention may be beneficial for treating cancer (e.g., breast cancer), and, indeed, may have one or more advantages or increased benefits when Compound A is used in combination another therapy, specifically a CDK4 inhibitor. For example, it is believed that the combination of Compound A and Compound B show improved safety and efficacy, potential to reduce side effects, including adverse events; potential to overcome resistance mechanisms, such as resistance to endocrine therapy; and potential to achieve similar or improved safety and efficacy through dose modification or dosing regimens, over the monotherapies of Compound A and Compound B alone.

### DEFINITIONS

Unless otherwise defined herein, scientific, and technical terms used in connection with the present invention have the meanings that are commonly understood by those of ordinary skill in the art.

The invention described herein suitably may be practiced in the absence of any element(s) not specifically disclosed herein.

As used herein, the singular form “a,” “an,” and “the” include plural references unless indicated otherwise. For example, “a” substituent includes one or more substituents.

As used herein, the term “about” when used to modify a numerically defined parameter (e.g., the dose of a compound) means that the parameter may vary by as much as 10% below or above the stated numerical value for that parameter. For example, a dose of about 5 mg means 5 mg±10%, i.e., it may vary from 4.5 mg to 5.5 mg.

As used herein, terms, including, but not limited to, “agent,” “composition,” “compound,” “drug,” and “therapeutic agent” may be used interchangeably to refer to compounds included in the methods and uses of the present disclosure.

As used herein, the terms, “subject,” “participant,” and “patient,” are used interchangeably, to refer to any animal, including mammals. Mammals according to the disclosure include canine, feline, bovine, caprine, equine, ovine, porcine, rodents, lagomorphs, primates, humans, and the like, and encompass mammals in utero. In embodiments, humans are suitable subjects. Human subjects may be of any gender and at any stage of development.

As used herein, the term “locally advanced or metastatic second line (2L) ER+HER2− breast cancer” refers to breast cancers in subjects who have received prior hormonal/endocrine therapy and chemotherapy in the locally advanced/metastatic setting and whose breast cancers have progressed.

Compound A, having the structure:

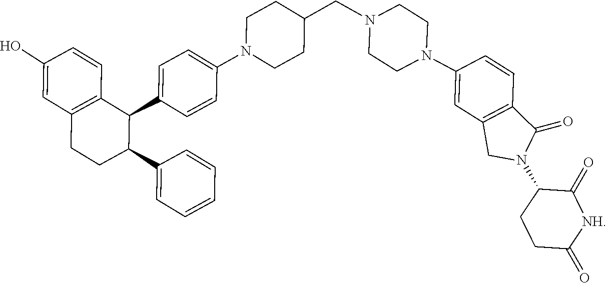 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Compound A is a Biopharmaceutics Classification System Class IV compound (low solubility/low permeability). Compound A can interconvert to its epimer, Compound C (or “Compd C”):

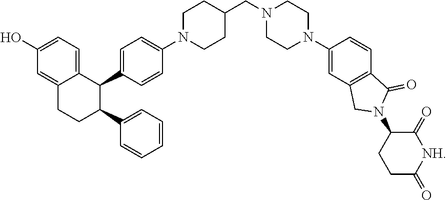 [SMILES: O=C1CC[C@@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Preclinical data demonstrates that the exposure of Compound C is limited compared to Compound A (<26%). Compound C does not degrade the estrogen receptor; however, Compound C shows similar antagonism of ER-dependent transcription compared to Compound A.

Cyclin-dependent kinases (CDKs) and related serine/threonine kinases are important cellular enzymes that perform essential functions in regulating cell division and proliferation. CDK inhibitors include pan-CDK inhibitors that target a broad spectrum of CDKs or selective CDK inhibitors that target specific CDK(s).

In embodiments, a selective CDK4 inhibitor of the present invention includes 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (Compound B):

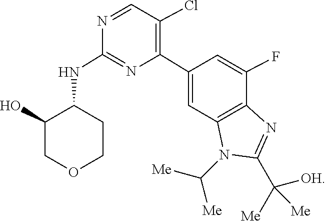 [SMILES: CC(C)n1c(C(C)(C)O)nc2c(F)cc(-c3nc(N[C@@H]4CCOC[C@H]4O)ncc3Cl)cc21]

or a solvate (e.g., hydrate) or a pharmaceutically acceptable salt thereof.

In embodiments, Compound B may exist in both unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm. The term ‘solvate’ is used herein to describe a molecular complex comprising the compound of the invention and one or more pharmaceutically acceptable solvent molecules, for example, ethanol. The term ‘hydrate’ is employed when the solvent is water. Pharmaceutically acceptable solvates in accordance with the invention include hydrates and solvates wherein the solvent of crystallization may be isotopically substituted, e.g., D₂O, d₆-acetone, d₆-DMSO.

Other embodiments also relate to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of the compounds described herein. Suitable base addition salts are formed from bases that form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine, and zinc salts.

The compounds described herein that are basic in nature can form a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds of the compounds described herein that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate, and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

### Combinations

Provided herein are methods for treating cancer comprising administering to a subject Compound A having a structure of:

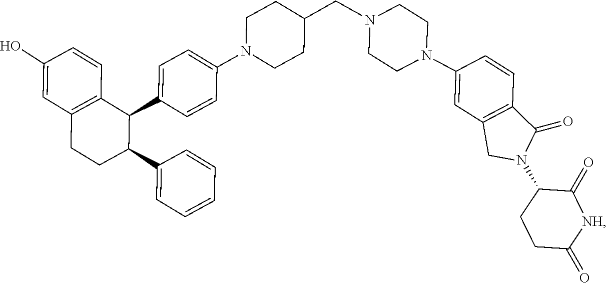 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, in combination with a selective CDK4 inhibitor.

In embodiments, a daily dose of compound having a structure of:

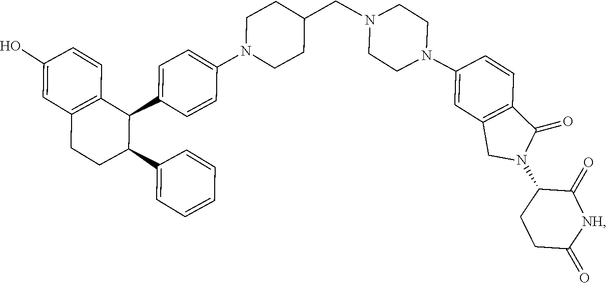 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is administered to the subject.

In embodiments, the daily dose of the compound having the structure:

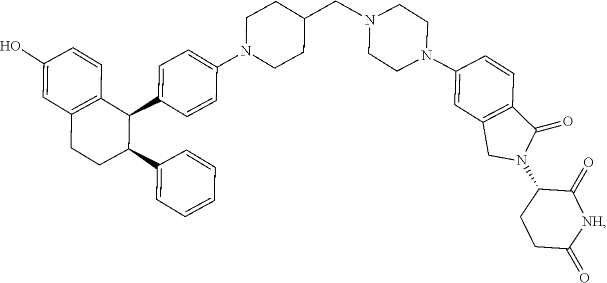 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is administered once a day (QD).

In embodiments, the daily dose of the compound having the structure:

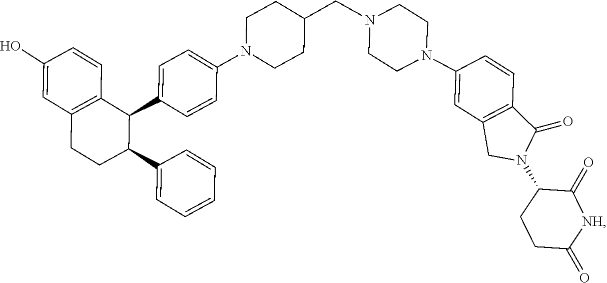 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is administered orally to the subject.

In embodiments, the subject is in a fed state.

In embodiments, the daily dose of the compound

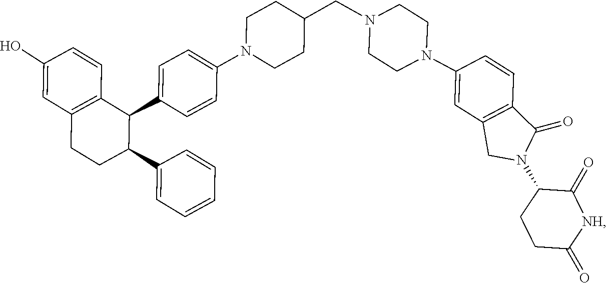 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is about 200 mg.

In embodiments, the daily dose of the compound

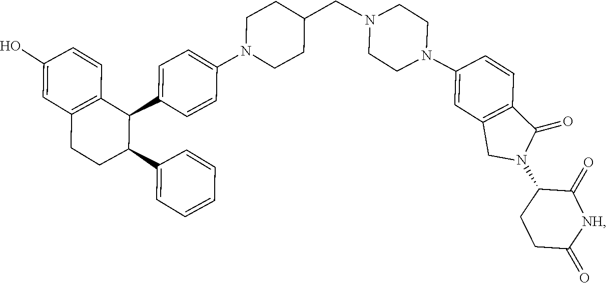 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is 200 mg.

In embodiments, the compound having the structure:

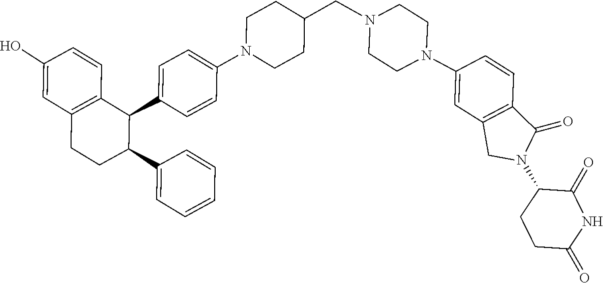 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is administered as a free base.

In some embodiments, the selective CDK4 inhibitor is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (Compound B), or a solvate (e.g., hydrate) or a pharmaceutically acceptable salt thereof.

In some embodiments, Compound A is administered daily in 28-day cycles. In certain embodiments, Compound B, or a pharmaceutically acceptable salt thereof, is administered orally twice a day for each 28-day cycle.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer.

In embodiments, the breast cancer is metastatic or locally advanced.

In embodiments, the breast cancer is estrogen receptor positive (ER+) breast cancer.

In embodiments, the estrogen receptor positive (ER+) breast cancer is human epidermal growth factor receptor 2 negative (HER2−).

In embodiments, the subject is human.

Also disclosed herein is Compound A having the structure:

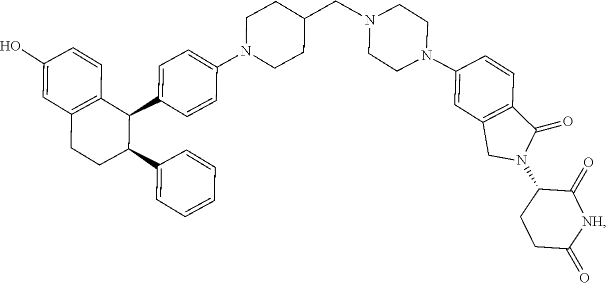 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt, for use according to any one of foregoing embodiments.

Also disclosed herein are uses of a compound having the structure:

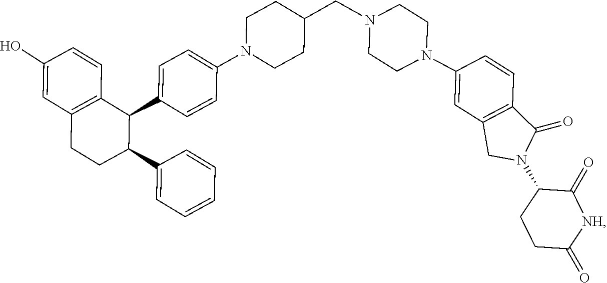 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt, in the manufacture of a medicament according to any one of foregoing embodiments.

Each of the embodiments described herein may be combined with any other embodiment(s) described herein not inconsistent with the embodiment(s) with which it is combined.

### Administration and Dosing

The terms “treat” and “treating” a cancer or a cancer-associated disease, as used herein, mean to administer a combination therapy according to the present disclosure to a subject, participant or patient having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The terms “treatment” and “therapy,” as used herein, unless otherwise indicated, refer to the act of treating as “treating” is defined immediately above. For the purposes of this disclosure, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. (2009) 50:1S-10S).

“Fed condition” or “fed state” as used to describe a subject herein, means that the subject has eaten less than 4 hours before a time point of interest, such as the time of administering Compound A. In embodiments, a subject in the fed state has not eaten for at most any of 4, 3, 2, 1, or 0.5 hours prior to administration of Compound A.

An “amount” for use and for treating a subject refers to an amount that provides, in single or multiple doses, in combination with one or more other agents, a detectable response of any duration of time (transient, medium, or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences, or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an agent in combination with one or more other agents, effective for producing a desired therapeutic effect upon administration to a subject, for example, to stem the growth, or result in the shrinkage, of a cancerous tumor. In reference to the treatment of cancer, a therapeutically effective amount refers to that amount that has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens also may be characterized as the ability to induce, enhance, maintain, or prolong disease control and/or overall survival in patients with these specific tumors, which may be measured as prolongation of the time before disease progression.

As used herein, “ameliorate” refers to any reduction in the extent, severity, frequency, and/or likelihood of a symptom or clinical sign characteristic of a particular disease. “Symptom” refers to any subjective evidence of disease or of a subject's condition.

As used herein, “daily dose” refers to a dose that is administered each day, such as an amount of Compound A that is administered each day or an amount of Compound B that is administered each day.

The daily dose or daily dosage of Compound A, or a pharmaceutically acceptable salt thereof, is the free base equivalent of Compound A. Dosage amounts provided herein refer to the dose of the free base form of Compound A, or are calculated as the free base equivalent of an administered Compound A salt form. For example, a dosage or amount of Compound A, such as about 100 mg or about 200 mg refers to the free base equivalent.

The daily dose or daily dosage of Compound B, or a pharmaceutically acceptable salt thereof, is the free base equivalent of Compound B. Dosage amounts provided herein refer to the dose of the free base form of Compound B, or are calculated as the free base equivalent of an administered Compound B salt form. For example, a dosage or amount of Compound B, such as about 100 mg or about 300 mg refers to the free base equivalent.

Embodiments of the present invention provide a dose, dosage, and dosing regimen comprising administering to a subject an amount, or an effective amount of Compound A, or a pharmaceutically acceptable salt thereof. The amount, or the therapeutically effective amount, can be a daily dose of about 200 mg. In another embodiment, a daily dose is 200 mg.

In embodiments, the daily dose of Compound A, or a pharmaceutically acceptable salt thereof, is administered once a day (QD).

The compounds disclosed herein may be administered orally. Oral administration may involve swallowing, so that the compound enters the gastrointestinal tract, or buccal or sublingual administration may be employed by which the compound enters the bloodstream directly from the mouth.

In embodiments, the daily dose of Compound A, or a pharmaceutically acceptable salt thereof, is administered orally.

Compound A, or a pharmaceutically acceptable salt thereof, may be present in a pharmaceutical composition, which includes a pharmaceutically acceptable excipient. A “pharmaceutically acceptable excipient” refers to a component that may be included in the compositions described herein, is physiologically suitable for pharmaceutical use, and causes no significant adverse effects nor therapeutic effects to a subject. The term “excipient” is used herein to describe any ingredient other than the compound(s) of the invention. The choice of excipient will to a large extent depend on factors such as the mode of administration, the effect of the excipient on solubility and stability, and the nature of the dosage form.

The compounds of the methods, uses, or combinations of the present invention may be formulated prior to administration. The formulation preferably will be adapted to the particular mode of administration. These compounds may be formulated with pharmaceutically acceptable excipients as known in the art and administered in a wide variety of dosage forms as known in the art. Dosage unit forms or pharmaceutical compositions suitable for oral administration include, but are not limited to tablets, capsules, such as gelatin capsules, pills, powders, granules, aqueous, and nonaqueous oral solutions and suspensions, packaged in containers adapted for subdivision into individual doses.

In embodiments, the daily dosage of Compound B, or a pharmaceutically acceptable salt thereof, is administered in the range of about 0.07 mg/day to about 7000 mg/day, and more commonly, of about 10 mg/day to about 1000 mg/day. Sometimes, the daily dosage is about 10 mg/day, about 20 mg/day, about 30 mg/day, about 40 mg/day, about 50 mg/day, about 60 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, about 700 mg/day, about 750 mg/day, about 800 mg/day, about 900 mg/day, or about 1000 mg/day. Sometimes, the daily dosage is about 10 mg/day to about 1000 mg/day, about 10 mg/day to about 750 mg/day, about 10 mg/day to about 600 mg/day, about 10 mg/day to about 300 mg/day, about 10 mg/day to about 150 mg/day, about 20 mg/day to about 750 mg/day, about 20 mg/day to about to 600 mg/day, about 20 mg/day to about to 300 mg/day, about 20 mg/day to about to 150 mg/day, about 50 mg/day to about 750 mg/day, about 50 mg/day to about 600 mg/day, about 50 mg/day to about 300 mg/day, about 50 mg/day to about 150 mg/day, about 75 mg/day to about 750 mg/day, about 75 mg/day to about 600 mg/day, about 75 mg/day to about 300 mg/day, or about 75 mg/day to about 150 mg/day.

Repetition of the administration or dosing regimens may be conducted as necessary to achieve the desired reduction or diminution of cancer cells. A “continuous dosing schedule,” as used herein, is an administration or dosing regimen without dose interruptions, e.g., without days off treatment. Repetition of 28-day treatment cycles without dose interruptions between the treatment cycles is an example of a continuous dosing schedule. In embodiments, the compounds of the combination of the present invention can be administered in a continuous dosing schedule. In embodiments, the compounds of the combination disclosed herein can be administered concurrently in a continuous dosing schedule.

In embodiments, Compound A is administered once daily to comprise a complete cycle of 28-days. Repetition of 28-day treatment cycles is continued during treatment in accordance with the methods and uses of the present disclosure.

Also disclosed herein are kits comprising the therapeutic agents of the combination of the present disclosure and written instructions for administration of the therapeutic agents. In embodiments, the written instructions elaborate and qualify the modes of administration of the therapeutic agents, for example, for simultaneous or sequential administration of the therapeutic agents of the present disclosure. In embodiments, the written instructions elaborate and qualify the modes of administration of the therapeutic agents, for example, by specifying the days of administration for each of the therapeutic agents during a 28-day treatment cycle.

### Methods of Treatment

In embodiments, provided herein are methods for treating cancer in a subject comprising administering to the subject an effective amount of Compound A as described herein in combination with an amount of a selective CDK4 inhibitor.

The term “combination,” or “combination therapy” as used herein, unless otherwise indicated, refers to the use of Compound A with one or more therapeutic agents, wherein Compound A and the one or more therapeutic agents are administered intermittently, concurrently, or sequentially, according to the same or different route of administration and according to the same or different dosage schedules.

The term “sequential” or “sequentially” refers to the administration of each therapeutic agent of the combination therapy of the invention, either alone or in a medicament, one after the other, wherein each therapeutic agent can be administered in any order. Sequential administration is particularly useful when the therapeutic agents in the combination therapy are in different dosage forms, for example, one agent is a tablet and another agent is a sterile liquid, and/or are administered according to different dosing schedules, for example, one agent is administered daily, and the second agent is administered less frequently such as weekly.

The term “concurrently” refers to the administration of each therapeutic agent in the combination therapy of the invention, either alone or in separate medicaments, wherein the second therapeutic agent is administered immediately after the first therapeutic agent, but that the therapeutic agents can be administered in any order. In a preferred embodiment the therapeutic agents are administered concurrently.

The term “intermittent” or “intermittently” refers to the administration of each therapeutic agent in the combination therapy of the invention, either alone or in separate medicaments, wherein one of the therapeutic agents is stopped and then restarted. Intermittent dosing may utilize regular “drug holidays” in which a patient is not exposed to drug.

The term “locally advanced,” as used herein, as it relates to cancer, may or may not be treated with curative intent. For example, locally advanced breast cancer (LABC) is defined by the U.S. National Comprehensive Cancer Network as a subset of breast cancer characterized by the most advanced breast tumors in the absence of distant metastasis, wherein the tumors are more than 5 cm in size with regional lymphadenopathy; tumors of any size with direct extension to the chest wall or skin, or both (including ulcer or satellite nodules), regardless of regional lymphadenopathy; presence of regional lymphadenopathy (clinically fixed or matted axillary lymph nodes, or any of infraclavicular, supraclavicular, or internal mammary lymphadenopathy) regardless of tumor stage. (Garg et al. Curr Oncol. 2015 October; 22(5): e409-10; National Comprehensive Cancer Network NCCN Clinical Practice Guidelines in Oncology: Breast Cancer. Fort Washington, PA: NCCN; 2015. Ver. 2.2015.)

The term “metastatic” as used herein, as it relates to cancer, cannot be treated with curative intent. For example, metastatic breast cancer refers to breast cancer that has spread beyond the breast and nearby lymph nodes to other parts of the body, e.g., bones, liver, lungs, brain. (www.cancer.org/cancer/breast-cancer.)

Those skilled in the art will be able to recognize and diagnose locally advanced and metastatic cancer in a patient or subject.

For convenience, certain well-known abbreviations, may be used herein, including: castration resistant prostate cancer (CRPC), estrogen receptor positive (ER+), human epidermal growth factor receptor 2 negative (HER2−), hormone receptor (HR), human epidermal growth factor receptor 2 positive (HER2+), non-small cell lung cancer (NSCLC), and progesterone receptor (PR).

In embodiments, the cancer is selected from lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, head and neck cancer, and combinations of two or more of the foregoing cancers.

Also disclosed herein are methods of treating cancer in a subject. In embodiments, the methods comprise treating cancer in a subject comprising administering to the subject an amount of the compounds described herein that are effective in treating the cancer.

In embodiments, the cancer is breast, lung, colon, brain, head and neck, prostate, stomach, pancreatic, ovarian, melanoma, endocrine, uterine, testicular, or bladder.

In embodiments, the cancer is breast, lung, prostate, pancreatic, or ovarian.

In embodiments, the cancer is breast, lung, or prostate.

In embodiments, the cancer is breast cancer.

In embodiments, the breast cancer is metastatic breast cancer.

In embodiments, the breast cancer is locally advanced breast cancer.

In embodiments, the breast cancer is HR+ breast cancer.

In embodiments, the HR+ breast cancer is PR+ and/or ER+ breast cancer.

In some embodiments, the breast cancer is PR+ breast cancer.

In embodiments, the breast cancer is ER+ breast cancer.

In embodiments, the breast cancer is ER+HER2− breast cancer.

In embodiments, the breast cancer is ER+HER2+ breast cancer.

In embodiments, the breast cancer is locally advanced or metastatic ER+ breast cancer.

In embodiments, the breast cancer is locally advanced or metastatic ER+HER2− breast cancer.

In embodiments, the breast cancer is locally advanced or metastatic ER+HER2+ breast cancer.

In embodiments, the breast cancer is metastatic, ER+, HER2− breast cancer.

In embodiments, the breast cancer is metastatic, ER+, HER2− breast cancer that is also locally advanced.

In embodiments, the lung cancer is non-small cell lung cancer.

In embodiments, the lung cancer is locally advanced or metastatic non-small cell lung cancer.

In embodiments, the prostate cancer is CRPC.

In embodiments, the prostate cancer is locally advanced or metastatic CRPC.

Also disclosed herein are methods of treating solid tumors in a subject. In embodiments, disclosed herein are methods of treating solid tumors in a subject comprising administering to the subject an amount of the compounds described herein that are effective in treating the solid tumor.

In embodiments, the solid tumor is breast, lung, colon, brain, head and neck, prostate, stomach, pancreatic, ovarian, melanoma, endocrine, uterine, testicular, or bladder.

In embodiments, the solid tumor is breast, lung, prostate, pancreatic, or ovarian.

In embodiments, the solid tumor is breast, lung, or prostate.

In embodiments, the solid tumor is breast cancer. For example, in certain embodiments the breast cancer is HR+ breast cancer. In other embodiments, the HR+ breast cancer is PR+ and/or ER+ breast cancer ER+ breast cancer.

In embodiments, the solid tumor is breast cancer. For example, in certain embodiments, the breast cancer is ER+HER2− breast cancer.

In embodiments, the solid tumor is breast cancer. For example, in certain embodiments, the breast cancer is ER+HER2+ breast cancer.

In embodiments, the solid tumor is breast cancer. For example, in certain embodiments, the breast cancer is locally advanced or metastatic ER+HER2− breast cancer.

In embodiments, the solid tumor is breast cancer. For example, in certain embodiments, the breast cancer is locally advanced or metastatic ER+HER2+ breast cancer.

In embodiments, the solid tumor is lung cancer. For example, in certain embodiments, the lung cancer is non-small cell lung cancer.

In embodiments, the solid tumor is lung cancer. For example, in certain embodiments, the lung cancer is locally advanced or metastatic non-small cell lung cancer.

In embodiments, the solid tumor is prostate cancer. For example, in certain embodiments, the prostate cancer is CRPC.

In embodiments, the solid tumor is prostate cancer. For example, in certain embodiments, the prostate cancer is locally advanced or metastatic castration resistant prostate cancer.

Also disclosed herein are methods of treating hematologic tumors in a subject. In certain embodiments, the method comprises treating hematologic tumors in a subject comprising administering to the subject an amount of the compounds described herein that is effective in treating the hematologic tumor.

In embodiments, the hematologic tumor is leukemia, lymphoma, or multiple myeloma.

In embodiments, the hematologic tumor is leukemia or lymphoma.

Also disclosed herein are methods of treating cancer in a subject with locally advanced or metastatic ER+HER2− breast cancer, CRPC, or NSCLC whose disease progressed on or is intolerant to standard therapy.

Also disclosed herein are methods of treating cancer in a subject with locally advanced or metastatic ER+HER2− breast cancer, CRPC, or NSCLC whose disease progressed on or is intolerant to standard therapy.

Also disclosed herein are methods of treating cancer in a subject with locally advanced or metastatic second line (2L) ER+HER2 breast cancer who have received prior hormonal/endocrine therapy and chemotherapy in the locally advanced/metastatic setting. In embodiments, the method comprises administering Compound A in combination with a selective CDK4 inhibitor to the subject.

Also disclosed herein are methods of treating cancer in a subject with locally advanced or metastatic 2L+ER+HER2 breast cancer who have received prior treatment with a CDK4/6 inhibitor. In embodiments, the method comprises administering Compound A in combination with a selective CDK4 inhibitor to the subject.

### Examples

In order that this invention may be better understood, the following examples are set forth. These examples are for purposes of illustration only and are not to be construed as limiting the scope of the invention in any manner.

### ABBREVIATIONS

AE: adverse event;
A/MBC: advanced or metastatic breast cancer;
ANCOVA: analysis of co-variance;
ATCC: american type culture collection;
AUC: area under curve;
AUP: animal usage protocol;
BID: twice a day, two times a day;
CBR: clinical benefit response;
CDK4: cyclin-dependent kinase 4;
CDK4/6: cyclin-dependent kinase 4/6;
CDX: cell-derived xenograft;
CL: clearance;
Cmax: maximum observed concentration;
CR: complete response;
CRD: clinical relevant dose; clinically relevant exposure level;
CTCAE: Common Terminology Criteria for Adverse Events;
ctDNA: circulating tumor deoxyribonucleic acid;
ΔT/ΔC: relative tumor volume change from baseline in treated group versus vehicle group;
ΔTζ: the mean tumor volume change obtained by subtracting the mean; tumor burden group on the first day of treatment (day 0) from the mean tumor burden on the assessment day;
DLT: dose limiting toxicity;
DoR: duration of response;
ECG: electrocardiogram;
ER: estrogen receptor;
FBS: fetal bovine serum;
HR+/HER2−: hormone receptor positive/human epidermal growth factor receptor 2 negative;
IACUC: Institutional Animal Care and Use Committee;
MC: methyl cellulose;
N: Number;
N/A: Not applicable;
NCI: National Cancer Institute;
OR: objective response;
OS: overall survival;
PD: progressive disease;
PFS: progression free survival;
PO: oral gavage;
PR: partial response;
RECIST: Response Evaluation Criteria in Solid Tumors;
RP2D: recommended phase 2 dose;
SD: stable disease;
PD: progressive disease;
PFS: progression free survival;
QD: once daily, once a day, once per day;
SC: subcutaneous;
SEM: standard error of the mean;
TGI: tumor growth inhibition;
Tmax: time to maximum concentration; and
TV: tumor volume.

### Example 1: Efficacy Evaluation of a Compound B in Combination with Compound A in a TGI Study in ER+HER2− Breast Cancer

### Abstract

The in vivo pharmacologic effects of a selective CDK4 inhibitor, Compound B, was evaluated in the xenograft tumor model MCF7 representing HR+/HER2− breast cancer. Compound B was tested using two doses (30 mg/kg, BID and 60 mg/kg, BID) both as a single agent and in combination with fulvestrant (at clinically relevant dose) and a novel PROTAC estrogen degrader Compound A. Compound B treatment as a single agent resulted in significant tumor growth inhibition and the combination with the estrogen receptor antagonist fulvestrant further improved the efficacy to stasis at 30 mg/kg and regression at 60 mg/kg. Compound A as a single agent at 30 mg/kg, QD resulted in significant tumor regression (119% TGI), therefore, the combination with Compound B at either 30 or 60 mg/kg did not result in an enhanced antitumor efficacy compared to Compound A treatment alone. Palbociclib in combination with Compound A was also tested and the results were not significantly different from Compound A treatment. Following cessation of treatment, regrowth of tumors was assessed across the treatment groups. The combination with Compound B (60 mg/kg, BID) plus Compound A demonstrated the most significant delay in tumor re-growth compared to other treatment groups. In the palbociclib plus Compound A treatment group the tumor regrowth was significantly slower compared to Compound A alone, but faster compared to Compound B (60 mg/kg, BID) plus Compound A. Overall, none of the combinations tested were significantly different from Compound A due to robust monotherapy TGI of Compound A, however, the assessment of tumor regrowth after treatment showed a significant growth delay with the Compound B plus Compound A combination compared to Compound A alone and palbociclib plus Compound A treatment groups.

### Rationale for Combination

CDK4/cyclin D1 pathway is highly activated in ER+ breast cancer and confers resistance to endocrine therapy
Cyclin D1/ER pathway crosstalk, drive ER breast cancer
Inhibition of CDK4/6 alone is insufficient to maintain long term G1 arrest
Blocking of cell cycle and ER pathways with CDK inhibitors+ER degrader is more efficient to prevent cell cycle entry

### Materials and Method

### Compound Formulation

Compound A was formulated as a suspension in 2% Tween 80: 98% PEG 400.

Compound B and palbociclib were formulated as a suspension in 0.5% methylcellulose A4M/water.

Fulvestrant was formulated as a suspension in 100% peanut oil.

### Animals

| Study Details | Information |
|---|---|
| System evaluated | Anti-tumor efficacy in MCF7 xenograft model |
| Species | Mus musculus |
| Strain / Catalog No. | NSG/NOD scid gamma #5557 |
| Gender and No. per | Female mice N = 10/group |
| group replicates | |
| Age | 6 to 8 weeks old |
| Source | Jackson Lab, Bar Harbor, ME USA (CDX) |

### Software

Data collection: StudyLog® (StudyLog® Systems, South San Francisco)
Graph plot: GraphPad Prism software 9.0.1 (GraphPad Software Inc, San Diego)
Data Analysis: TGI Analyzer ANCOVA Analysis

### Cell Culture

To generate MCF7 PO donors, cells were obtained from ATCC and cultured in RPM11640 supplemented with 10% FBS. All cells were maintained in a humidified incubator at 37 °C with 5% CO2.

### Cell Implant and Compound Treatment

To establish the MCF7 PO donor mice, cells (5×10⁶/mouse with 50% Cultrex® Basement Membrane Matrix) were subcutaneously (in 0.2 mL total volume) implanted in female NSG mice supplemented with 17β-ESTRADIOL water ad libidum (8.5 μg/mL concentration). The 17β-ESTRADIOL water supplement was administered about 7 days before the implant and all throughout the duration of the study.

To establish the MCF7 P1, when tumor volume of MCF7 PO donor mice reached a range of 700 to 800 mm3, tumors were collected and cut into fragments. The fragments were subsequently transplanted into secondary recipient mice for TGI study expansion. Secondary recipient mice were than randomly assigned to groups (n=10) when tumor volumes reached a range between 143 mm3 and 209 mm3 and subsequently treated with: 1) vehicle (0.5% methylcellulose A4M/water), PO; 2) fulvestrant at 10 mg/kg twice in the first week, then weekly afterwards, SC; 3) Compound A at 30 mg/kg QD, PO; 4) Compound B at 30 mg/kg BID, PO; 5) Compound B at 60 mg/kg BID, PO; 6) Compound B at 30 mg/kg plus fulvestrant; 7) Compound B at 60 mg/kg plus fulvestrant; 8) Compound B at 30 mg/kg plus Compound A; 9) Compound B at 60 mg/kg plus ARV-47; 10) palbociclib at 10 mg/kg BID, PO plus Compound A. All mice received treatment continuously until Day 42. TGI was assessed on Day 41 post first dose.

### In Vivo Efficacy Evaluation and Statistical Analysis

For the efficacy measurements, tumor volume and body weights were collected twice a week. Tumor volume was calculated using the [(Length×Width×Width)/2)] formula. TGI was calculated as 100*(1−ΔT/ΔC). The ΔC (ΔT) was obtained by subtracting the mean tumor burden in the vehicle (treated) group on the first day of treatment (Day 0) from the mean tumor burden in vehicle (treated) group on the assessment day. Statistical analysis was performed using TGI Analyzer ANCOVA. BLISS statistical analysis (statistical modeling of single drug response from drug combination data) was used to measure synergy effect, a combination is synergistic if the tumor volume is less than the BLISS with pvalue of <0.05.

### Results and Discussion

1. Antitumor Efficacy of Compound B in Combination with Compound A and Fulvestrant in HR+HER2− BC Model MCF7

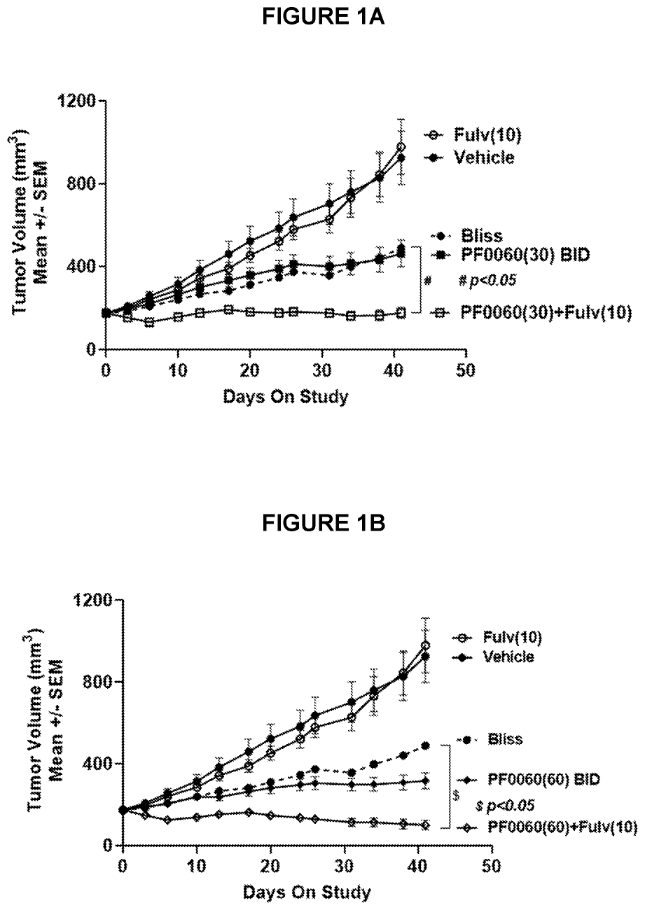

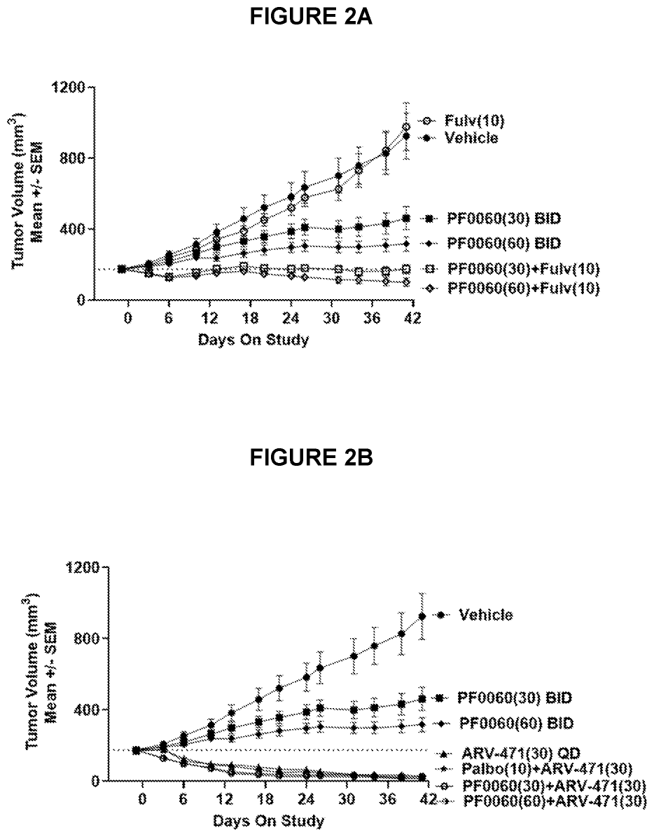

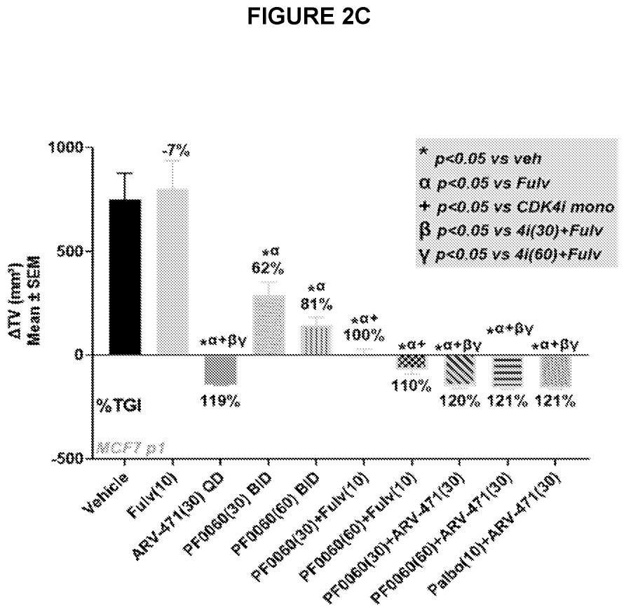

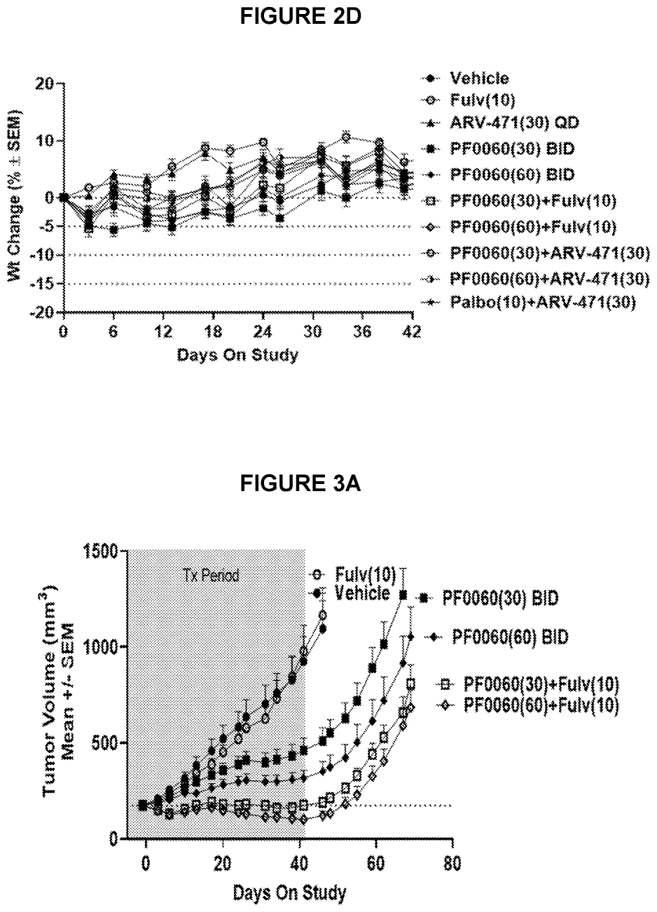

Compound B was tested at two CRD dose levels (30 mg/kg, BID and 60 mg/kg, BID) as a single agent or in combination with Compound A at 30 mg/kg QD or fulvestrant at CRD (10 mg/kg). Palbociclib at CRD (10 mg/kg, BID) in combination with Compound A served as a positive comparator. On Day 41 after the dosing initiation, Compound B single agent treatment at two doses (30 mg/kg, BID and 60 mg/kg, BID) showed significant efficacy (TGI at 62% and 81% respectively) versus vehicle (p<0.05). The single agent activity of fulvestrant showed no tumor growth inhibition (−7% TGI) while single agent activity of Compound A led to a significant regression (TGI at 119%). The combination of fulvestrant with Compound B at either 30 or 60 mg/kg BID displayed a dose dependent synergistic (as measured by BLISS analysis; FIGS. 1A and 1B, respectively) effect (TGI at 100% and 110%, respectively) (FIGS. 2A and 2C, respectively), while the combination with Compound A showed no combinatorial benefit over Compound A monotherapy at the dose level tested (TGI of 120% and 121% respectively) (FIGS. 2B and 2C, respectively). Positive control comparator palbociclib plus Compound A also showed no combinatorial benefit (TGI at 121%) and was not significantly different from either of the two dose levels of Compound B in combination with Compound A (Table 1). Comparing palbociclib plus Compound A to Compound B (30 mg/kg and 60 mg/kg, BID) plus fulvestrant, the combination of palbociclib to Compound A showed significantly more robust response. Comparing Compound B plus fulvestrant to Compound B plus Compound A, the combination of Compound B plus Compound A was significantly more efficacious, however, when comparing Compound B plus Compound A to single agent Compound A, there was no significant difference (Table 1).

There were no adverse effects observed throughout the treatment period, as assessed by body weight loss (FIG. 2D).

TABLE 1
Dosing Regimen and the Antitumor Efficacy of Compound
B in Combination With Compound A or Fulvestrant in
HR+ HER2− BC Model MCF7

| | | | | BLISS |
|---|---|---|---|---|
| | Treatment | | | Combination |
| Group | (mg/kg) | Regimen | TGI % | Analysis |
| 1 | Vehicle | PO, BID × 42 D | 0.0 | — |
| 2 | Fulvestrant | SC, QD D 1, D 3, | −7 | — |
| | (10) | 1x/week × 5 W | | |
| 3 | Compd A (30) | PO, QD × 42 D | 119*α+βγ | — |
| 4 | Compd B (30) | PO, BID × 42 D | 62*α | — |
| 5 | Compd B (60) | PO, BID × 42 D | 81*+α | — |
| 6 | Compd B (30) + | BID + QD | 100*+α | # |
| | Fulvestrant | D 1, D 3, | | |
| | (10) | 1x/week × 42 D | | |
| 7 | Compd B (60) + | BID + QD | 110*+α | $ |
| | Fulvestrant | D 1, D 3, | | |
| | (10) | 1x/week × 42 D | | |
| 9 | Compd B (60) + | BID + QD × | 121*α+βγ | — |
| | Compd A (30) | 42 D | | |
| 10 | Palbociclib | BID + QD × | 121*α+βγ | — |
| | (10) + | 42 D | | |
| | Compd A (30) | | | |

TGI was assessed on Day 41 post first dose. n = 10 in all groups except Group 8 where n = 9 (lost 1 mouse, unknown cause). Statistical analysis was performed using ANCOVA.
*indicates p < 0.05 versus vehicle;
αindicates p < 0.05 versus fulvestrant;
+indicates p < 0.05 versus Compound B single agents;
βindicates p < 0.05 versus Compound B (30) + fulvestrant;
γindicates p < 0.05 versus Compound B (60) + fulvestrant.
ANCOVA BLISS combination analysis was run to measure synergistic effect.
#: indicates synergistic effect (group 4 versus group 6), p < 0.05 vs. BLISS;
$: indicates synergistic effect (group 5 versus group 7), p < 0.05 vs. BLISS.

2. Tumor Regrowth Assessment after Treatment with Compound B in Combination with Compound A in HR+HER2− BC Xenograft Model MCF7

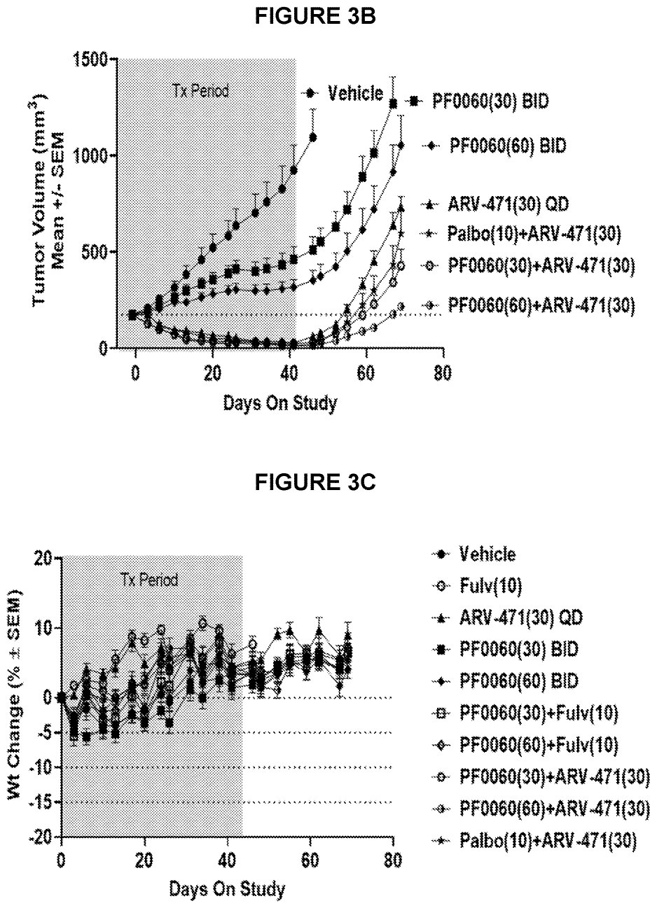

Tumor growth delay was monitored after treatment was discontinued on Day 43 and statistical analysis was performed on Day 26 post last dose (Day 69 post study start). Tumor stasis was not sustained in the combination groups of Compound B (30 mg/kg or 60 mg/kg, BID) plus fulvestrant (FIG. 3A), nor was tumor regression maintained in the Compound A single agent, Compound B (30 mg/kg or 60 mg/kg, BID) plus Compound A combinations, or palbociclib plus Compound A (FIG. 3B). During the treatment period, there were no significant differences in the TGI observed with Compound A single agent, Compound B (30 mg/kg and 60 mg/kg, BID) plus Compound A, or palbociclib (10 mg/kg, BID) plus Compound A groups, however, when treatment ended there were significant differences in regrowth of the tumors. The combination of Compound B (30 mg/kg, BID or 60 mg/kg, BID) with Compound A showed a significant dose dependent growth delay compared to Compound A single agent, Compound B single agent at the two doses (30 mg/kg and 60 mg/kg, BID), Compound B (30 mg/kg and 60 mg/kg, BID) plus fulvestrant and palbociclib plus Compound A (Table 2). Comparing the regrowth of palbociclib plus Compound A to Compound B plus fulvestrant, the regrowth of palbociclib plus Compound A was significantly slower compared to the lower dose (Compound B 30 mg/kg, BID plus fulvestrant) but not at the higher dose (Compound B 60 mg/kg, BID plus fulvestrant).

There was no significant body weight loss seen throughout the post treatment period (FIG. 3C).

TABLE 2
Statistical Analysis of the Tumor Regrowth Post Treatment
With Compound B in Combination with Compound A or
Fulvestrant in HR+ HER2− BC model MCF7

| | | | ANCOVA |
|---|---|---|---|
| | | | Analysis |
| | | | (Day 26 |
| | Treatment | | post end of |
| Group | (mg/kg) | Regimen | treatment) |
| 1 | Vehicle | PO, BID × 42 D | — |
| 2 | Fulvestrant (10) | SC, QD D 1,D 3, | — |
| | | 1x/week × 5 W | |
| 3 | Compd A (30) | PO, QD × 42 D | β |
| 4 | Compd B (30) | PO, BID × 42 D | — |
| 5 | Compd B (60) | PO, BID × 42 D | — |
| 6 | Compd B (30) + | BID + QD D 1, D 3, | β |
| | Fulvestrant (10) | 1x/week × 42 D | |
| 7 | Compd B (60) + | BID + QD D 1, D 3, | β |
| | Fulvestrant (10) | 1x/week × 42 D | |
| 8 | Compd B (30) + | BID + QD × 42 D | &βγ |
| | Compd A (30) | | |
| 9 | Compd B (60) + | BID + QD × 42 D | &β*+$ |
| | Compd A (30) | | |
| 10 | Palbociclib (10) + | BID + QD × 42 D | &βγ |
| | Compd A (30) | | |

Regrowth analysis was assessed 26 days post end of treatment. Statistical analysis was performed using ANCOVA.
&: indicates p < 0.05 versus Compound A (10);
β: indicates p < 0.05 versus Compound B single agent;
γ: indicates p < 0.05 versus Compound B (30) + fulvestrant (10);
*: indicates p < 0.05 versus Compound B (60) + fulvestrant (10);
+: indicates p < 0.05 versus Compound B (30) + Compound A (10);
$: indicates p < 0.05 versus palbociclib (10) + Compound A (10).

### CONCLUSIONS

In conclusion, during treatment period, the combination benefit of Compound A with Compound B at both dose levels (120% and 121% TGI, respectively) could not be assessed due to robust single agent activity of Compound A (119% TGI). In addition the combination of fulvestrant with Compound B at both dose levels resulted in a dose dependent synergistic effect when compared to the monotherapies. The assessment of tumor volume after treatment ended showed a significant dose dependent growth delay with the combination of Compound B plus Compound A. In addition, combination of Compound A with the higher dose of Compound B (60 mg/kg, BID) had the most robust tumor growth delay compared to any other combination tested. The tumor growth delay of palbociclib plus Compound A was significantly better than Compound A monotherapy and the lower dose of Compound B (30 mg/kg, BID) plus fulvestrant, but was comparable to the lower dose combination of Compound B (30 mg/kg, BID) plus Compound A.

Compound A+Compound B showed significantly longer tumor regrowth delay versus: 1) monotherapy groups; 2) palbociclib+Compound A; and 3) Compound B+fulvestrant.

### Example 2: An Interventional Safety and Efficacy Phase 1b/2, Open-Label Study to Investigate Tolerability, Pharmocokinetic (PK), and Antitumor Activity of Compound A in Combination with Compound B in Participants with ER+/HER2− Advanced or Metastatic Breast Cancer

### Overview

This study is an open-label, multi-center, Phase 1b/2 study to evaluate the safety, antitumor activity, and PK of Compound A in combination with Compound B in the participants with Estrogen Receptor Positive/Human Epidermal Growth Factor Receptor 2 Negative (ER+/HER2−) advanced or metastatic breast cancer.

### Brief Plain Language Summary of Study

The purpose of this study is to learn about the safety and effects of giving Compound A along with Compound B. Compound A is studied to see if can be a possible treatment for advanced metastatic breast cancer. This type of cancer would have spread from where it started (breast) to other parts of the body and would be tough to treat. The study is seeking participants who have breast cancer that:
is hard to treat (advanced) and may have spread to other organs (metastatic).
is sensitive to hormonal therapy (it is called estrogen receptor positive).
is no longer responding to treatments taken before starting this study.

All the participants will receive Compound A and Compound B. Both medicines will be taken by mouth. The medicines will be taken at home. The experience of people receiving the study medicines will be studied. This will help see if the study medicines are safe and effective. Participants will continue to take Compound A and Compound B until:
their cancer is no longer responding, or
side effects become too severe.

### Brief Plain Language Summary of Study Design

Phase 1b will use an escalation/de-escalation approach to determine the recommended phase 2 dose (RP2D) of Compound A when administered in combination with Compound B. The decision to escalate the starting dose level of Compound B will be using mTPI-2 decision criteria based on the number of dose limiting toxicity (DLT)-evaluable participants and the number of DLTs in those participants during the DLT observation period (Cycle 1). Phase 2 will further evaluate the preliminary antitumor activity and safety of the combination RP2D.

### Design:

The combination will be assessed in two parts in this study:
Phase 1b: dose escalation/de-escalation phase to evaluate the safety and tolerability of Compound A in combination with Compound B and select up to 2 recommended doses for dose expansion for the combination (combination recommended doses for expansion [RDEs]) via the modified toxicity probability interval (mTPI)-2 dose finding design.
Phase 2: if at least 2 dose levels from Phase 1b (e.g., two combination RDEs) are considered tolerable, a randomized dose optimization Phase 2 will be conducted to further evaluate the safety, tolerability, PK, and antitumor activity of Compound A in combination with Compound B to determine the recommended Phase 2 dose (RP2D) of the combination. If only one dose level from Phase 1b is considered tolerable, a single arm Phase 2 dose expansion will be conducted.

Phase 1b: In Phase 1b the starting dose level (DL1) of Compound A will be 200 mg once daily (QD) and the starting dose level of Compound B will be 100 mg twice daily (BID). The decision to escalate the starting dose level of Compound B will be using mTPI-2 decision criteria based on the number of DLT-evaluable participants and the number of DLTs in those participants during the DLT observation period. DL2 will be the combination of Compound A 200 mg QD and Compound B 300 mg BID. Based on the mTPI-2 decision criteria and depending on the occurrence of DLTs and need for dose reduction, two additional dose levels may be investigated: Compound A 100 mg QD in combination with Compound B 100 mg BID and/or Compound A 100 mg QD in combination with Compound B 300 mg BID.

A minimum of 3 DLT-evaluable participants will be required in each cohort at a given dose level before a decision is made to de-escalate, escalate, or remain at the current dose level. Additional participants will be enrolled in a specific cohort to replace participants who are not considered DLT-evaluable.

Phase 2: In the Phase 2 portion of the study up to two RDEs will be further explored.

If at least 2 dose levels from Phase 1b are considered tolerable, a randomized 1:1 dose optimization Phase 2 will be conducted and approximately 25 participants for each arm will be enrolled and treated. If only one dose level from Phase 1b is considered tolerable, a single arm Phase 2 will be conducted in approximately 25 participants.

The RP2D of Compound A and Compound B, identified as the dose that can be used in further large-scale testing, will be determined based on an overall assessment of safety, tolerability, PK and early signals of efficacy. The RP2D may be the same as the maximum tolerated dose (MTD) or RDE, or may be lower. The RP2D will be determined in this study after the Phase 2 expansion cohort(s) have provided sufficient data on which to make such a determination.

### Number of Participants

The total sample size is estimated to be approximately 65 participants for Phase 1b and Phase 2. The sample size is estimated to be approximately 15 participants for Phase 1b and 50 participants (approximately 25 per dose level for Phase 2).

### Objectives, Endpoints and Estimands

The objectives, endpoints and estimands of the Phase 1b portion of the clinical study are shown below in Table 4.

TABLE 4

| Objectives | Endpoints | Estimands |
|---|---|---|
| Primary: | Primary: | Primary: |
| To assess safety and | DLTs during DLT | DLT rate estimated |
| tolerability of Compd | observation period | based on data from |
| A in combination with | (Cycle 1). | DLT-evaluable |
| Compd B in | | participants during |
| participants | | the DLT observation |
| with ER+/HER2− | | period (Cycle 1). |
| A/MBC to select up to | | |
| 2 RDEs for the | | |
| combination. | | |
| Secondary: | Secondary: | Secondary: |
| To evaluate the overall | AEs as characterized by | Not applicable. |
| safety profile. | type, frequency, | |
| | intensity (as graded by | |
| | NCI CTCAE version | |
| | 5.0), seriousness, and | |
| | relationship to Compd A | |
| | in combination with | |
| | Compd B. | |
| | Incidence of laboratory | |
| | abnormalities. | |
| | Incidence of ECG | |
| | abnormalities. | |
| To evaluate antitumor | Confirmed OR (CR or | Not applicable. |
| activity of Compd A | PR) by investigator | |
| in combination with | assessment. | |
| Compd B. | DoR by investigator | |
| | assessment. | |
| | CBR (confirmed CR or | |
| | PR at any time, or | |
| | SD ≥ 24 weeks) by | |
| | investigator | |
| | assessment. | |
| | PFS by investigator | |
| | assessment. | |
| To evaluate the | Plasma concentrations | Not applicable. |
| plasma exposure of | of Compd A, Compd C, | |
| Compd A, Compd C, | and Compd B. | |
| and Compd B when | Steady-state Cmax, | |
| Compd A and Compd | Tmax, and AUClast of | |
| B are given in | Compd A, Compd C, | |
| combination. | and Compd B. | |
| | If data permit CL/F, | |
| | Vz/F, and t1/2 of Compd | |
| | A, Compd C, and | |
| | Compd B. | |

The objectives, endpoints and estimands of the Phase 2 portion of the clinical study are shown below in Table 5.

TABLE 5

| Objectives | Endpoints | Estimands |
|---|---|---|
| Primary: | Primary: | Primary: |
| To assess the clinical | Confirmed OR (CR or | The treatment effect |
| antitumor activity of | PR) determined by | of Compd A in |
| Compd A in | investigator | combination with |
| combination with | assessment. | Compd B assessed |
| Compd B. | | by the OR rate based |
| | | on Investigator |
| | | assessment per |
| | | RECIST v1.1 in the |
| | | analysis population. |
| | | Participants who do |
| | | not have a post- |
| | | baseline disease |
| | | assessment due to |
| | | early PD, who |
| | | receive anticancer |
| | | therapies other than |
| | | the study intervention |
| | | prior to achieving an |
| | | objective response, |
| | | or who die, |
| | | experience |
| | | documented PD, or |
| | | stop disease |
| | | assessments for any |
| | | reason prior to |
| | | achieving an |
| | | objective response |
| | | will be counted as |
| | | nonresponders. |
| Secondary: | Secondary: | Secondary: |
| To determine | DoR by investigator | Not applicable. |
| additional | assessment. | |
| antitumor activity | CBR (confirmed CR | |
| outcomes of Compd | or PR at any time | |
| A in combination | or SD ≥ 24 weeks) | |
| with Compd B. | by investigator | |
| | assessment. | |
| | PFS by investigator | |
| | assessment. | |
| | OS. | |
| To further characterize | AEs as characterized | Not applicable |
| the overall safety | by type, frequency, | |
| profile and | intensity (as graded by | |
| tolerability | NCI CTCAE version | |
| of Compd A | 5.0), seriousness, and | |
| in combination | relationship to Compd | |
| with Compd B. | A in combination with | |
| | Compd B. | |
| | Incidence of laboratory | |
| | abnormalities. | |
| | Incidence of ECG | |
| | abnormalities. | |
| To evaluate the plasma | Plasma concentrations | Not applicable |
| exposure of Compd A, | of Compd A, Compd C, | |
| Compd C and Compd | and Compd B. | |
| B when Compd A and | | |
| Compd B are given in | | |
| combination. | | |
| To assess changes | ctDNA plasma | Not applicable |
| from baseline levels in | quantitative changes | |
| plasma ctDNA with | from pre-treatment to | |
| treatment. | evaluate potential | |
| | predictability of their | |
| | associations with clinical | |
| | outcomes. | |

The arms of the clinical study are shown below in Table 6.

TABLE 6

| Arm Title | Type | Description |
|---|---|---|
| Compound A in | Experimental | Compound A administered |
| combination | | orally once daily (QD) |
| with Compound B | | continuously and Compound |
| | | B administered orally twice |
| | | daily (BID) continuously on |
| | | 28-day cycles. |

The interventions of the clinical study are shown below in Table 7.

TABLE 7

| Intervention | | Associated | | Other |
|---|---|---|---|---|
| Name | Type | Arm | Description | Names |
| Compound | Drug | Compound A in | Daily oral | Vepdegestrant |
| A | | combination with | dosages of | ARV-471 |
| | | Compound B | Compound A | PF-07850327 |
| | | | continuously, | |
| | | | dose | |
| | | | escalation/de- | |
| | | | escalation in | |
| | | | Phase 1b until | |
| | | | recommended | |
| | | | phase 2 dose | |
| | | | (RP2D) | |
| | | | determined, | |
| | | | cycles lasting | |
| | | | 28 days | |
| Compound | Drug | Compound A in | Daily oral | PF-07220060 |
| B | | combination with | dosages of | |
| | | Compound B | Compound B | |
| | | | continuously, | |
| | | | dose | |
| | | | escalation/de- | |
| | | | escalation in | |
| | | | Phase 1b until | |
| | | | recommended | |
| | | | phase 2 dose | |
| | | | (RP2D) | |
| | | | determined, | |
| | | | cycles 28 days | |

The primary and secondary outcome measures of the clinical study are provided below.

### Primary Outcome Measures:

Phase 1b: Number of Participants With Dose Limiting Toxicities [Time Frame: 28 days] Dose Limiting Toxicities (DLTs) rate for Compound A in combination with Compound B, estimated based on data from DLT-evaluable participants during the DLT observation period (Cycle 1).
Phase 2: Percentage of Participants With Objective Response by investigator assessment [Time Frame: Up to approximately 1 year]
   Objective Response (OR) refers to confirmed Complete Response (CR) or Partial Response (PR) according to Response Evaluation Criteria in Solid Tumor (RECIST) version 1.1. as determined by investigator assessment.

### Secondary Outcome Measures:

Phase 1b and Phase 2: Evaluation of Safety of Compound A in combination with Compound B (number of participants experiencing any AE, SAE, treatment-related AE and treatment-related SAE) [Time Frame: First study drug dose through a minimum of 28 Days After Last study drug administration]
   An adverse event (AE) is any untoward medical occurrence in a participant, temporally associated with the use of study treatment, whether or not considered related to the study treatment. A severe adverse event (SAE) is any untoward medical occurrence that, at any dose: resulted in death; required inpatient hospitalization or prolongation of existing hospitalization: was life-threatening; resulted in persistent or significant disability/incapacity; congenital anomaly/birth defect and other important medical events. A treatment-related AE is any untoward medical occurrence attributed to study drug in a participant who received study drug.
   AEs were graded by the investigator according to the CTCAE version 5.0 and coded using MedDRA where reported, Grade 1=mild; Grade 2=moderate; Grade 3=severe; Grade 4=life-threatening or disabling; and Grade 5=death.
a Phase 1b and Phase 2: Evaluation of Safety of Compound A in combination with Compound B (number of participants with lab abnormalities—Hematology and coagulation parameters) [Time Frame: First study drug dose through a minimum of 28 Days After Last study drug administration]
   Blood samples were collected for the analysis of the following hematology and coagulation parameters: hemoglobin [g,L], platelets, leukocytes, neutrophils, lymphocytes, monocytes, eosinophils and basophils [10\hat{ }9/L]; partial thromboplastin time prolonged and prothrombin time [seconds]; international normalized ratio increased. Number of participants with hematological and coagulation abnormalities by grade as per Common Terminology Criteria for Adverse Events (CTCAE version 5.0) were reported. Grade 1=mild; Grade 2=moderate; Grade 3=severe: Grade 4=life-threatening or disabling; and Grade 5=death. For laboratory tests without CTCAE grade definitions, results will be categorized as normal, abnormal, or not done.
a Phase 1b and Phase 2: Evaluation of Safety of Compound A in combination with Compound B (number of participants with lab abnormalities—chemistry parameters) [Time Frame: First study drug dose through a minimum of 28 Days After Last study drug administration]
   Blood samples were collected for analysis of clinical chemistry parameters. These included: alanine aminotransferase, alkaline phosphatase, aspartate aminotransferase, [international unit per liter (IU/L)]; Lipase and amilase [IU/L](limited to cycle 1 only); Albumin, bilirubin, urea, calcium, creatinine, glucose, magnesium, phosphate, uric acid chloride, potassium and sodium [millimol per liter (mmol/L)]; eGFR [milliliter per minute (ml/min)]1 Number of participants with hematological and coagulation abnormalities by grade as per Common Terminology Criteria for Adverse Events (CTCAE version 5.0) were reported. Grade 1=mild; Grade 2=moderate; Grade 3=severe: Grade 4=life-threatening or disabling; and Grade 5=death. For laboratory tests without CTCAE grade definitions, results will be categorized as normal, abnormal, or not done,
Phase 1b and Phase 2: Evaluation of Safety of Compound A in combination with Compound B (number of participants with changes from baseline for ECG parameters) [Time Frame: First study drug dose through a minimum of 28 Days After Last study drug administration]
   The following ECG parameters were analyzed and changes from baseline were assessed: heart rate, PR interval, QT interval, QRS interval and QT interval corrected using Fridericia's formula (QTcF).
Phase 1b and Phase 2: Evaluation of Tolerability of Compound A in combination with Compound B (number of participants experiencing any AE, SAE, treatment-related AE and treatment-related SAE) [Time Frame: First study drug dose through a minimum of 28 Days After Last study drug administration]
   An adverse event (AE) is any untoward medical occurrence in a participant, temporally associated with the use of study treatment, whether or not considered related to the study treatment. A severe adverse event (SAE) is any untoward medical occurrence that, at any dose: resulted in death; required inpatient hospitalization or prolongation of existing hospitalization; was life-threatening; resulted in persistent or significant disability/incapacity: congenital anomaly/birth defect and other important medical events. A treatment-related AE is any untoward medical occurrence attributed to study drug in a participant who received study drug.
   AEs were graded by the investigator according to the CTCAE version 5.0 and coded using MedDRA where reported. Grade 1=mild; Grade 2=moderate; Grade 3=severe; Grade 4=life-threatening or disabling; and Grade 5=death.
Phase 1b and Phase 2: Evaluation of Tolerability of Compound A in combination with Compound B (number of participants with lab abnormalities—Hematology and coagulation parameters) [Time Frame: First study drug dose through a minimum of 28 Days After Last study drug administration]
   Blood samples were collected for the analysis of following hematology and coagulation parameters: hemoglobin [g/L], platelets, leukocytes, neutrophils, lymphocytes, monocytes, eosinophils and basophils [10^{A}9/L]: partial thromboplastin time prolonged and prothrombin time [seconds]; international normalized ratio increased. Number of participants with hematological and coagulation abnormalities by grade as per Common Terminology Criteria for Adverse Events (CTCAE version 5.0) were reported. Grade 1=mild; Grade 2=moderate: Grade 3=severe: Grade 4=life-threatening or disabling; and Grade 5=death. For laboratory tests without CTCAE grade definitions, results will be categorized as normal, abnormal, or not done.
a Phase 1b and Phase 2: Evaluation of Tolerability of Compound A in combination with Compound B (number of participants with lab abnormalities ~chemistry parameters) [Time Frame: First study drug dose through a minimum of 28 Days After Last study drug administration]
   Blood samples were collected for analysis of clinical chemistry parameters. These included: alanine aminotransferase, alkaline phosphatase, aspartate aminotransferase, [international unit per liter (IU/L)]; Lipase and amilase [IU/L](limited to cycle 1 only); Albumin, bilirubin, urea, calcium, creatinine, glucose, magnesium, phosphate, uric acid chloride, potassium and sodium [millimol per liter (mmol/L)]; eGFR [milliliter per minute (ml/min)], Number of participants with hematological and coagulation abnormalities by grade as per Common Terminology Criteria for Adverse Events (CTCAE version 5.0) were reported. Grade 1=mild; Grade 2=moderate; Grade 3=severe; Grade 4=life-threatening or disabling; and Grade 5=death. For laboratory tests without CTCAE grade definitions, results will be categorized as normal, abnormal, or not done.
a Phase 1b and Phase 2: Evaluation of Tolerability of Compound A in combination with Compound B (number of participants with changes from baseline for ECG parameters) [Time Frame: First study drug dose through a minimum of 28 Days After Last study drug administration]
   Following ECG parameters were analyzed and changes from baseline were assessed: heart rate, PR interval, QT interval, QRS interval and QT interval corrected using Fridericia's formula (QTcF).
Phase 1b: To evaluate antitumor activity of Compound A in combination with Compound B [Time Frame: Up to approximately 1 year]
   Objective response (OR) refers to confirmed complete response (CR) or partial response (PR) according to Response Evaluation Criteria in Solid Tumor (RECIST) version 1.1. as determined by investigator assessment.
Phase 1b and Phase 2: Duration of Response by investigator assessment. [Time Frame: Up to approximately 1 year]
   Duration of Response (DoR) is defined for participants with confirmed OR (CR or PR) as the time from the first documentation of OR to the first documentation of objective tumor progression or to death due to any cause, whichever occurs first.
a Phase 1b and Phase2: Percentage of participants with Clinical Benefit Response by investigator assessment. [Time Frame: Up to approximately 1 year] Clinical Benefit Response (CBR) is defined as the proportion of participants with Best Overall Response of confirmed CR or PR at any time, or Stable Disease (SD) ≥24 weeks
a Phase 1b and Phase 2: Progression Free Survival by investigator assessment. [Time Frame: Up to approximately 1 year]
   Progression Free Survival (PFS) is defined as the time from the date of first dose of study interventions to the date of first documentation of PD or death due to any cause, whichever occurs first.
a Phase 1b and Phase 2: Plasma concentrations of Compound A, Compound C and Compound B when given in combination. [Time Frame: Phase 1b: Pre-dose Day 8; Pre-, 0.5, 1, 2, 4, 6, 8, 12 h post-dose Day 15; Pre- and 4-3 h post-dose 29; Pre- and 4-8 h post-dose Day 43: Pre-dose Days 57, 113 and 169, Phase 2: Pre- and 4-8 h post-dose Days 15, 29 and 43; Pre-dose Days 57, 113 and 169]
   To evaluate the plasma exposure of Compound A, Compound C, and Compound B when Compound A and Compound B are given in combination.
a Phase 1b: Evaluation of the PK of Compound A and Compound B when given in combination [Time Frame: Phase 1b (a cycle is 28 days): Pre-dose on Day 8 of Cycle 1; Pre-dose, 0.5, 1, 2, 4, 6, 8, 12 h post-dose on Day 15 of Cycle 1; Pre-dose and 4-8 h on Day 1 of Cycle 2; Pre-dose and 4-8 h post-dose on Day 15 of Cycle 2; Pre-dose on Day 1 of Cycles 3, 5 and 7]
   Steady-state Cmax (maximum observed serum concentration of drug [Micrograms per milliliter]) of Compound A, Compound C, and Compound B.
Phase 1b: Evaluation of the PK of Compound A and Compound B when given in combination [Time Frame: Phase 1b (a cycle is 23 days): Pre-dose on Day 8 of Cycle 1; Pre-dose, 0.5, 1, 2, 4, 6, 3, 12 h post-dose on Day 15 of Cycle 1: Pre-dose and 4-8 h on Day 1 of Cycle 2; Pre-dose and 4-8 h post-dose on Day 15 of Cycle 2; Pre-dose on Day 1 of Cycles 3, 5 and 7]
   Steady-state Tmax (time taken (in hours) to reach the maximum serum drug concentration) of Compound A, Compound C, and Compound B.
Phase 1b: Evaluation of the PK of Compound A and Compound B when given in combination [Time Frame: Phase 1b (a cycle is 28 days): Pre-dose on Day 8 of Cycle 1; Pre-dose, 0.5, 1, 2, 4, 6, 8, 12 h post-dose on Day 15 of Cycle 1: Pre-dose and 4-Sh on Day 1 of Cycle 2; Pre-dose and 4-Sh post-dose on Day 15 of Cycle 2; Pre-dose on Day 1 of Cycles 3, 5 and 7]
   Steady-state AUClast (Area under the plasma concentration time-curve from zero to the last measured concentration (AUClast) of Compound A, Compound C, and Compound B,
Phase 2: ctDNA plasma quantitative changes from pre-treatment [Time Frame: Phase 2 (each cycle is 28 days): Pre-dose on Day 1 of Cycles 1, 2 and 3 and after last treatment administration]
   To assess changes from baseline levels in plasma circulating tumor DNA (ctDNA) with treatment and to evaluate potential predictability of their associations with clinical outcomes.

### Eligibility:

| | |
|---|---|
| Sex: | All |
| Gender Based: | No |
| Accepts Healthy Volunteers: | No |

### Inclusion Criteria

Participants must meet the following inclusion criteria to be eligible for enrollment into the study:
1. Participants aged 18 years or older (or the minimum age of consent in accordance with local regulations) at screening.
2. Histological or cytological diagnosis of breast cancer. At time of enrollment this must not be amenable to surgical resection with curative intent (≥1% ER+stained cells as per local practice on the most recent tumor biopsy HER2− tumor by immunohistochemistry (IHC) or in-situ hybridization per ASCO/CAP).
   Participants who have bilateral breast cancers that are both ER+/HER2− are eligible.
   Tumor block collected at the time of diagnosis with local recurrent or metastatic disease or archival tumor tissue is required for inclusion (Phase 1b and Phase 2).
3. Prior anticancer therapies:
Phase 1b:
   Participants should have received at least 1 line of standard of care (SOC) for A/MBC.
   Prior fulvestrant allowed.
   ≥1 prior chemotherapy line (no antibody-drug conjugates permitted) for A/MBC setting allowed.
Phase 2:
   At least one and maximum 2 lines of endocrine therapy (ET) in A/MBC setting and most recent ET-based regimen for >6 months.
   1, and only 1, prior CDK4/6 inhibitor, inhibitor-based regimen required (independent of the setting e.g., adjuvant or advanced/metastatic).
   Up to 1 prior regimen of cytotoxic chemotherapy (no antibody-drug conjugates permitted) in the A/MBC setting.
   Prior fulvestrant allowed.
4. Lesions at study entry:
Phase 1b:
   Participant with only non-measurable lesion (including skin or bone lesion only) are eligible.
Phase 2:
   Participants must have at least 1 measurable lesion as defined by RECIST v1.1. Participants with bone lesions only can be included if at least one bone lesion has a measurable component as for RECIST v1.1.
5. Eastern Cooperative Oncology Group (ECOG) performance status (PS) at study entry:
   Phase 1b: ECOG PS 0 or 1.
   Phase 2: ECOG PS ≤2.

### Exclusion Criteria

Participants with any of the following characteristics/conditions will be excluded:
1. Participants in visceral crisis at risk of life-threatening complications in the short term, including participants with massive uncontrolled effusions (pleural, pericardial, and/or peritoneal), pulmonary lymphangitis, and liver involvement >50%.
2. Participants with newly diagnosed brain metastases, or symptomatic central nervous system (CNS) metastases, carcinomatous meningitis, or leptomeningeal disease as indicated by clinical symptoms, cerebral edema, and/or progressive growth. Participants with a history of CNS metastases or cord compression are eligible if they have been definitively treated (e.g., radiotherapy, stereotactic surgery) and clinically stable (including participants with residual CNS symptoms/deficits) and discontinued anti-seizure medications and corticosteroids or at least 28 days prior to first dose of investigational medicinal product (IMP).
3. Refractory nausea and vomiting, chronic gastrointestinal (GI) disease, GI ulcer, GI bleeding, active inflammatory bowel disease, inability to swallow the formulated product, or previous, significant gastric-bowel resection that would preclude adequate absorption of study interventions.
4. History of any other malignancies within the past three years, except for the following: (1) adequately treated basal or squamous cell carcinoma of the skin; (2) curatively treated in situ carcinoma of the cervix. All other malignancies must have been curatively treated with no evidence of disease for >3 years. Participants with inflammatory breast cancer are excluded.
5. Impaired cardiovascular function or clinically significant cardiovascular diseases, as defined as:
   Any of the following in the previous 6 months: myocardial infarction, severe/unstable angina, coronary/peripheral artery bypass graft, symptomatic congestive heart failure (New York Heart Association class Ill or IV), cerebrovascular accident, transient ischemic attack, or symptomatic pulmonary embolism or other clinically significant episode of thromboembolic disease.
   Symptomatic cardiac valve disease. Participants with mitral valve prolapse that is asymptomatic or not associated with clinically significant sequalae (e.g., mitral regurgitation) are eligible.
   Corrected QT-Fridericia method (QTcF) >470 msec at screening or any of the following in the previous 6 months: congenital long QT syndrome, Torsade de Pointes, clinically important arrhythmias, left anterior hemiblock (bifascicular block), ongoing cardiac dysrhythmias of Grade >2, atrial fibrillation of any grade.
6. The following prior procedures and treatment are not permitted:
   Anticancer systemic therapies:
   Phase 1b and Phase 2:
      Prior therapy with selective estrogen receptor down regulators (SERDs) (excluding fulvestrant), selective estrogen receptor modulators (SERMs) (excluding tamoxifen), selective estrogen receptor covalent antagonists (SERCAs), complete estrogen receptor antagonists (CERANs), and Proteolysis Targeting Chimeras (PROTACs) (approved or experimental), or elacestrant.
   Major surgery, radiotherapy, prior endocrine therapy, CDK4/6 inhibitors, or other anticancer treatments within 514 days prior to first dose of IMP (for anticancer therapy containing an antibody-based agents, approved or investigational, 28 days or 5 half-lives, whichever is shorter).
   Participants who received: prior radiotherapy to >25% of bone marrow, or prior hematopoietic stem cell, or bone marrow transplantation are not eligible independent of when it was received.
7. Any unresolved toxicities from prior surgeries or therapies Grade >1 at the time of study enrollment except for Grade 2 alopecia, peripheral neuropathy, arthralgia, or other toxicities not considered a safety risk for the participant per the investigator's judgment.
8. Any medical or psychiatric condition including recent (within the past year) or active suicidal ideation/behavior or laboratory abnormality that may increase the risk of study participation or, in the investigator's judgment, make the participant inappropriate for the study, or any social situations that would limit study compliance.
9. Previous administration with an investigational product (drug or vaccine) within 30 days (or as determined by the local requirement) or 5 half-lives preceding the first dose of study intervention used in this study (whichever is longer). Participation in studies of other investigational products (drug or vaccine) at any time during their participation in this study.
10. Concurrent administration of medications, food, or herb supplements that are strong inhibitors/inducers of cytochrome P (CYP)3A or UGT2B7, and drugs known to predispose to Torsade de Pointes or QT interval prolongation. Prior use of strong inhibitors of CYP3A or UGT2B7 must be stopped 7 days, strong inducers of CYP3A or UGT2B7 must be stopped 14 days before enrollment in the study.
11. Renal impairment defined by an estimated Glomerular Filtration Rate (eGFR)<45 mL/min/*. In equivocal cases, a 24-hour urine collection test can be used to estimate the creatinine clearance more accurately.
   Use the 2021 CKD-EPI eGFR (serum creatinine only) equation to calculate eGFR (in units of mL/min/1.73 m²). By multiplication of ([eGFR]×[nomogram-based participant body surface area (m2))=estimated creatinine clearance (eCrCl) in mL/min.
12. Hepatic dysfunction defined as:
   Total bilirubin 1.5×upper limit of normal (ULN) unless the participant has documented Gilbert's syndrome (in this case total bilirubin >3×ULN);
   Aspartate aminotransferase (AST) >3×ULN (>5×ULN if attributed to liver metastases)
   Alanine aminotransferase (ALT) >3×ULN (>5×ULN if attributed to liver metastases)
   Alkaline phosphatase 0.2.5×ULN or >5×ULN if liver or bone metastases present.
   aPTT >1.25×ULN and international normalized ratio (INR) >1.25 unless the participant is receiving anticoagulation, then activated partial thromboplastin time (aPTT) and INR should be within the therapeutic range of the intended use.
13. Hematologic abnormalities defined as:
   Absolute neutrophil count (ANC)<1,500/mm3 or 1.5×109/L;
   Platelets <100,000/mm³ or 100×109/L;
   Hemoglobin <9 g/dL. Receipt of a blood transfusion (blood or blood products) within 14 days before the first dose is allowed.
14. Known active infection including hepatitis B virus, hepatitis C virus, and human immunodeficiency virus (HIV) or acquired immunodeficiency syndrome (AIDS)-related illness (screening for chronic conditions is not required). A participant who has a positive test result for severe acute respiratory syndrome coronavirus 2 (SARS-CoV-2) infection, is known to have asymptomatic infection, or is suspected of having SARS-CoV-2, is not eligible for the study, except in case SARS-CoV-2 infection occurring during the screening period is resolved at least 14 days prior to enrollment.
Note: active viral hepatitis infection is defined as untreated/uncontrolled hepatitis (e.g., hepatitis B patients who are treated and whose liver function tests (LFTs) meet eligibility are not considered to have active hepatitis B). For HIV infection, the study participant is eligible if the disease is controlled with treatment, and the therapy does not exclude the participant as per Exclusion criterion #10.

We claim:

1. A method for treating cancer comprising administering to a subject Compound A having a structure of: 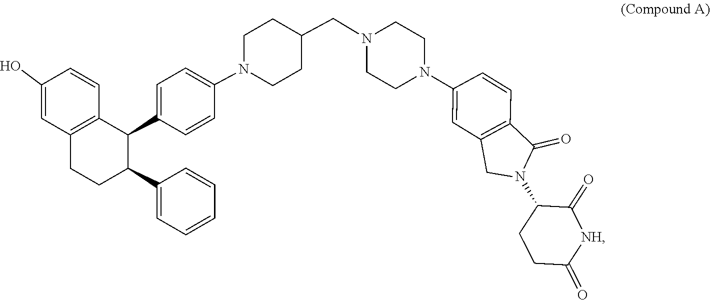 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, in combination with a selective CDK4 inhibitor.

2. The method of claim 1, wherein a daily dose of Compound A, or a pharmaceutically acceptable salt thereof, is administered to the subject.

3. The method of claim 2, wherein the daily dose of Compound A, or a pharmaceutically acceptable salt thereof, is about 200 mg.

4. The method of claim 2, wherein the daily dose of Compound A, or a pharmaceutically acceptable salt thereof, is about 100 mg.

5. The method of any one of claims 1 to 4, wherein the daily dose of Compound A, or a pharmaceutically acceptable salt thereof, is administered once a day (QD).

6. The method of any one of claims 1 to 5, wherein the selective CDK4 inhibitor is administered concurrently or sequentially.

7. The method of any one of claims 1 to 6, wherein Compound A, or a pharmaceutically acceptable salt thereof, is administered daily in 28-day cycles and the selective CDK4 inhibitor, is administered once a day or twice a day for each 28-day cycle.

8. The method of any one of claims 1 to 6, wherein the selective CDK4 inhibitor is administered intermittently.

9. The method of any one of claims 1 to 8, wherein the daily dose of Compound A, or a pharmaceutically acceptable salt thereof, is administered orally to the subject.

10. The method of any one of claims 1 to 9, wherein the subject is in a fed state.

11. The method of any one of claims 1 to 10, wherein the selective CDK4 inhibitor is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof.

12. The method of claim 11, wherein a daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is administered to the subject.

13. The method of claim 12, wherein the daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is about 100 mg.

14. The method of claim 12, wherein the daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is about 300 mg.

15. The method of claim 13 or claim 14, wherein the daily dose of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a solvate (e.g., hydrate) or a pharmaceutically acceptable salt thereof, is administered twice daily (BID).

16. The method of any one of claims 1 to 15, wherein the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

17. The method of claim 16, wherein the cancer is breast cancer, lung cancer, prostate cancer, pancreatic cancer, or ovarian cancer.

18. The method of claim 17, wherein the cancer is breast cancer, lung cancer, or prostate cancer.

19. The method of claim 18, wherein the cancer is breast cancer.

20. The method of claim 19, wherein the breast cancer is metastatic or locally advanced.

21. The method of claim 18 or 19, wherein the breast cancer is estrogen receptor positive (ER+) breast cancer.

22. The method of claim 21, wherein the estrogen receptor positive (ER+) breast cancer is human epidermal growth factor receptor 2 negative (HER2−).

23. The method of any one of claims 1 to 22, wherein the subject is human.
